SEQUENCE LISTING (4 sequences)

SEQ ID NO: 1 - RNA, 19 nt, AEQUORIA VICTORIA
cuacaacagccacaacguc

SEQ ID NO: 2 - RNA, 19 nt, AEQUORIA VICTORIA
caacaucucgacaccagca

SEQ ID NO: 3 - RNA, 19 nt, AEQUORIA VICTORIA
cagccacaacgucuauauc

SEQ ID NO: 4 - RNA, 19 nt, AEQUORIA VICTORIA
acagaccaccgugucuaac

(19) United States
(12) Patent Application Publication
(10) Pub. No.: US 2004/0248164 A1
(43) Pub. Date: Dec. 9, 2004

(54) RNAi arrays and methods for using the same

(76) Inventors: Jen-Tsan Chi, Mountain View, CA (US); Howard Y. Chang, Burlingame, CA (US); Patrick Brown, Stanford, CA (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/794,589

(22) Filed: Mar. 4, 2004

Related U.S. Application Data
(60) Provisional application No. 60/452,449, filed on Mar. 5, 2003.

(51) Int. Cl. C12Q 1/68

(52) U.S. Cl. 435/6

(57) ABSTRACT

RNAi arrays and methods for using the same are provided. The subject arrays are characterized by having two or more distinct RNAi agents. The arrays find use in methods where cells are contacted with the arrays and the activity of the RNAi agents is determined by evaluating the contacted cells. The subject arrays and methods find use in a variety of applications, such as high throughput loss of function genomic applications.

### CROSS-REFERENCE TO RELATED APPLICATIONS

This application claims priority (pursuant to 35 U.S.C. § 119(e)) to the filing date of the U.S. Provisional Patent Application Ser. No. 60/452,449 filed Mar. 5, 2003; the disclosure of which is herein incorporated by reference.

### INTRODUCTION

1. Field of the Invention

The field of this invention is RNA interference.

2. Background of the Invention

Double-stranded RNA induces potent and specific gene silencing through a process referred to as RNA interference (RNAi) or posttranscriptional gene silencing (PTGS). RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. For a review of the RNAi process, see Paddison & Hannon, Cancer Cell (2002) 2:17-23.

RNAi has become the method of choice for loss-of-function investigations in numerous systems, including C. elegans, Drosophila, fungi, plants, and even mammalian cell lines. In such assays, RNAi agents corresponding to the gene of interest, e.g., synthetic double stranded siRNA molecules having a sequence homologous to a sequence found in a target mRNA transcribed from the gene of interest, are introduced into a cell that contains the gene of interest and the phenotype of the cell is then determined. Any deviation in observed phenotype to the control wild type phenotype is then used as a determination of function of the gene of interest, since the observed phenotype results from the siRNA mediated inactivation of the gene of interest.

As more and more genes and their sequences are identified, of particular interest in RNAi loss-of-function investigations is the development of high throughput formats for such assays, where a plurality of distinct RNAi agents are assayed simultaneously for their effect on gene function.

Relevant Literature

Published U.S. Application No. 20020006664. Published PCT applications of interest include WO 01/68836 and WO 03/010180.

### SUMMARY OF THE INVENTION

RNAi arrays and methods for using the same are provided. The subject arrays are characterized by having two or more distinct RNAi agents immobilized on the surface of a substrate. The arrays find use in methods where cells are contacted with the arrays and the activity of the RNAi agents is determined by evaluating the contacted cells. The subject arrays and methods find use in a variety of applications, such as high throughput functional (e.g., loss of function) genomic applications.

### BRIEF DESCRIPTION OF THE FIGURES

FIG. 1. Test of transitive RNAi in HEK293 cells. FIG. 1A—Experimental strategy for transitive RNAi. The square indicates the original trigger siRNA, and the dashed lines indicate secondary siRNAs. Effect of siRNAs on expression of GFP fusion constructs. HEK293 cells were transfected with the indicated constructs and siRNAs and photographed by fluorescence microscopy 48 hours after transfection. FIG. 1B—Effect of siRNAs on luciferase-actin expression. Luciferase activity in cells transfected with the indicated constructs and siRNAs are shown; the values shown are the mean+standard deviation of triplicate experiments.

FIG. 2. siRNA microarray for gene silencing. (A) Experimental strategy for siRNA microarray. The desired cDNA and siRNAs are printed as individual spots on glass slides and exposed briefly to lipid before placing HEK293 cells on the printed slides in culture dish. Transfected cells are visualized using fluorescent microscopy and evaluated for the effect of RNAi. Parallel RNAi on microarrays. Fluorescence photomicrograph of cells were taken after reverse transfection of the indicated siRNA and cDNAs.

### DEFINITIONS

For convenience, certain terms employed in the specification, examples, and appended claims are collected here.

As used herein, the term “vector” refers to a nucleic acid molecule capable of transporting another nucleic acid to which it has been linked. One type of vector is a genomic integrated vector, or “integrated vector”, which can become integrated into the chromosomal DNA of the host cell. Another type of vector is an episomal vector, i.e., a nucleic acid capable of extra-chromosomal replication in an appropriate host, e.g., a eukaryotic or prokaryotic host cell. Vectors capable of directing the expression of genes to which they are operatively linked are referred to herein as “expression vectors”. In the present specification, “plasmid” and “vector” are used interchangeably unless otherwise dear from the context.

As used herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides.

As used, herein, the term “gene” or “recombinant gene” refers to a nucleic acid comprising an open reading frame encoding a polypeptide of the present invention, including both exon and (optionally) intron sequences. A “recombinant gene” refers to nucleic acid encoding such regulatory polypeptides, that may optionally include intron sequences that are derived from chromosomal DNA. The term “intron” refers to a DNA sequence present in a given gene that is not translated into protein and is generally found between exons. As used herein, the term “transfection” means the introduction of a nucleic acid, e.g., an expression vector, into a recipient cell by nucleic acid-mediated gene transfer.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and even synthetic DNA sequences. A transcription termination sequence will usually be located 3′ to the coding sequence.

Likewise, “encodes”, unless evident from its context, will be meant to include DNA sequences that encode a polypeptide, as the term is typically used, as well as DNA sequences that are transcribed into inhibitory antisense molecules.

The term “loss-of-function”, as it refers to genes inhibited by the subject RNAi method, refers a diminishment in the level of expression of a gene (e.g., reducing expression of a gene) when compared to the level in the absence of the RNAi agent, i.e., in a cell not transfected by the RNAi agent By reducing expression is meant that the level of expression of a target gene or coding sequence is reduced or inhibited by at least about 2-fold, usually by at least about 5-fold, e.g., 10-fold, 15-fold, 20-fold; 50-fold, 100-fold or more, as compared to a control. By modulating expression of a target gene is meant altering, e.g., reducing, transcription/translation of a coding sequence, e.g., genomic DNA, mRNA etc., into a polypeptide, e.g., protein, product.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the terms “transduction” and “transfection” are art recognized and mean the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a dsRNA construct.

“Transient transfection” refers to cases where exogenous DNA does not integrate into the genome of a transfected cell, e.g., where episomal DNA is transcribed into mRNA and translated into protein.

A cell has been “stably transfected” with a nucleic acid construct when the nucleic acid construct is capable of being inherited by daughter cells.

As used herein, a “reporter gene construct” is a nucleic acid that includes a “reporter gene” operatively linked to at least one transcriptional regulatory sequence. Transcription of the reporter gene is controlled by these sequences to which they are linked. The activity of at least one or more of these control sequences can be directly or indirectly regulated by the target receptor protein. Exemplary transcriptional control sequences are promoter sequences. A reporter gene is meant to include a promoter-reporter gene construct that is heterologously expressed in a cell.

As used herein, “transformed cells” refers to cells that have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control. For purposes of this invention, the terms “transformed phenotype of malignant mammalian cells” and “transformed phenotype” are intended to encompass, but not be limited to, any of the following phenotypic traits associated with cellular transformation of mammalian cells: immortalization, morphological or growth transformation, and tumorigenicity, as detected by prolonged growth in cell culture, growth in semi-solid media, or tumorigenic growth in immuno-incompetent or syngeneic animals.

As used herein, “proliferating” and “proliferation” refer to cells undergoing mitosis.

As used herein, “immortalized cells” refers to cells that have been altered via chemical, genetic, and/or recombinant means such that the cells have the ability to grow through an indefinite number of divisions in culture.

The “growth state” of a cell refers to the rate of proliferation of the cell and the state of differentiation of the cell.

“Inhibition of gene expression” refers to the absence (or observable decrease) in the level of protein and/or mRNA product from a target gene. “Specificity” refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western bloffing, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. Lower doses of administered active agent and longer times after administration of active agent may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target mRNA or translation of target protein. As an example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

### DESCRIPTION OF THE SPECIFIC EMBODIMENTS

RNAi arrays and methods for using the same are provided. The subject arrays are characterized by having two or more distinct RNAi agents. The arrays find use in methods where cells are contacted with the arrays and the activity of the RNAi agents is determined by evaluating the contacted cells. The subject arrays and methods find use in a variety of applications, such as high throughput functional genomic (e.g., loss of function) applications.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended, claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, representative methods, devices and materials are now described.

All publications mentioned herein are incorporated herein by reference for the purpose of describing and disclosing the components that are described in the publications that might be used in connection with the presently described invention.

As summarized above, the subject invention is directed to RNAi arrays and methods for using the same, e.g., in high throughput loss-of-function assays. In further describing the subject invention, the subject RNAi arrays are described first in greater detail, followed by a review of representative methods of using the subject assays, as well as kits that include the subject arrays.

RNAi Arrays

As indicated above, the subject invention provides RNAi arrays. By RNAi array is meant a composition of matter that includes two or more different RNAi agents positioned in known locations on a surface of a substrate. As such, the subject arrays include a plurality of distinct or different RNAi agents immobilized on a surface of a substrate. By plurality is meant at least 2, usually at least about 10, and more usually at least about 25, where the number of different RNAi agents in the array may be much greater, being at least about 500 or more, such as at least about 1000 or more, at least about 5000 or more, at least about 10,000 or more etc.,.

By RNAi agent is meant an agent that modulates expression of a target gene by a RNA interference mechanism. The RNAi agents are, in certain embodiments, small ribonucleic acid molecules (also referred to herein as interfering ribonucleic acids), i.e., oligoribonucleotides, that are present in duplex structures, e.g., two distinct oligoribonucleotides hybridized to each other or a single ribo-oligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt.

Where the RNA agent is a duplex structure of two distinct ribonucleic acids hybridized to each other, e.g., an siRNA (such as d-siRNA as described in copending application Ser. No. 60/377,704; the disclosure of which is herein incorporated by reference), the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides.

The weight of the RNAi agents of this embodiment typically ranges from about 5,000 daltons to about 35,000 daltons, and in many embodiments is at least about 10,000 daltons and less than about 27,500 daltons, often less than about 25,000 daltons.

As mentioned above, the arrays include two or more different RNAi agents. A feature of the RNAi agents is that they correspond to a gene. An RNAi agent corresponds to a given gene if it includes a sequence (e.g., of at least about 10 nt in length, such as at least about 15 nt in length, including at least about 20 nt in length or longer, such as 21 nt in length, 22 nt in length, etc.) of nucleotides that is homologous to a sequence found in the gene to which it corresponds. In many embodiments, a given siRNA agent and its corresponding gene include sequences that hybridized to each other under stringent conditions, as defined above.

The arrays may include a single RNAi agent for a given gene, such that each RNAi agent on the array corresponds to a different gene, i.e., has a different cognate gene, or a given gene may have two or more different corresponding RNAi agents on the array. Where multiple RNAi agents are present for a given gene on the array, the number of RNAi agents that correspond to a given gene may range from about 2 to about 100 or more, such as from about 2 to about 50 or more, including from about 2 to about 25 or more.

The RNAi agents displayed on the array may be directed to genes of known or unknown function, where in many embodiments the RNAi agents may be directed genes of unknown function, e.g., in embodiments where the arrays are employed in loss-of-function assays. As such, the RNAi agents may correspond to gene sequences found in libraries of ESTs, etc.

As mentioned above, the two or more RNAi agents of the subject arrays (in many embodiments microarrays) are present on the surface of a substrate. A variety of solid supports or substrates are suitable for use as substrates of the invention, including both flexible and rigid substrates. By flexible is meant that the support is capable of being bent, folded or similarly manipulated without breakage. Examples of flexible solid supports include acrylamide, nylon, nitrocellulose, polypropylene, polyester films, such as polyethylene terephthalate, etc.

In contrast, rigid supports do not readily bend, and include glass, fused silica, quartz; plastics,. e.g. polytetrafluoroethylene, polypropylene, polystyrene, polycarbonate, and blends thereof, and the like; metals, e.g. gold, platinum, silver, and the like; etc.

Derivitized and coated slides are of interest in certain embodiments. Such slides are commercially available, or may be produced using conventional methods. For example, SuperAldehyde™ substrates contain primary aldehyde groups attached covalently to a glass surface. Coated-slides include films of nitrocellulose (FastSlides™, Schleicher & Schuell), positively-charged nylon membranes (CastSlides™, Schleicher & Schuell, and a polyacrylamide matrix (HydroGel™, Packard Bioscience), etc.

The substrates can take a variety of configurations, including filters, fibers, membranes, beads, particles, dipsticks, sheets, rods, etc., usually a planar or planar three-dimensional geometry is preferred. The materials from which the substrate can be fabricated should ideally exhibit a low level of non-specific binding during binding events, except for specific cases, in which some non-specific binding is preferred.

Representative array formats are further described in U.S. application Publication No. 20020006664; the disclosure of which is herein incorporated by reference.

In one embodiment of the invention, the substrate comprises a planar surface, and the RNAi agents are spotted on the surface in an array. The RNAi spots on the substrate can be any convenient shape, but will often be circular, elliptoid, oval or some other analogously curved shape. The local density of the spots on the solid surface can be at least about 500/cm² and usually at least about 1000/cm² but does not exceed about 10,000/cm², and usually does not exceed about 5000/cm², in many embodiments. The spot to spot distance (center to center) is usually from about 100 μm to about 200 μm. The spots can be arranged in any convenient pattern across or over the surface of the support, such as in rows and columns so as to form a grid, in a circular pattern, and the like, where generally the pattern of spots will be present in the form of a grid across the surface of the solid support.

The subject substrates can be prepared using any convenient means. One means of preparing the supports is to synthesize the RNAi agents, and then deposit the pre-synthesized agents as a spot on the support surface. The RNAi agents can be prepared using any convenient methodology, such as by chemical synthesis, in vitro transcription, the method described in copending application Ser. No. 60/377,704 (the disclosure of which is herein incorporated by reference); etc.

The prepared RNAi agents can then be spotted on the support using any convenient methodology, including manual techniques, e.g. by micro pipette, ink jet, pins, etc., and automated protocols. Of particular interest is the use of an automated spotting device, such as the Beckman Biomek 2000 (Beckman Instruments). A number of contact and non-contact microarray printers are available and may be used to print the binding members on a substrate. For example, non-contact printers are available from Perkin Elmer (BioChip Arrayer™, Packard). Contact printers are commercially available from TeleChem International (Arraylt™). Non-contact printers are of particular interest because they are more compatible with soft/flexible surfaces.

In one embodiment of the method, referred to as a “gelatin” embodiment, an RNAi-containing mixture, referred to herein as a gelatin-RNAi mixture, comprises RNAi agent and gelatin, which is present in an appropriate solvent, such as water or double deionized water. The mixture is spotted onto a surface, such as a slide, thus producing a surface bearing (having affixed thereto) the gelatin-RNAi mixture in defined locations. The resulting product is allowed to dry sufficiently that the spotted gelatin-RNAi mixture is affixed to the slide and the spots remain in the locations to which they have become affixed, under the conditions used for subsequent steps in the method. For example, a mixture of RNAi in gelatin is spotted onto a slide, such as a glass slide coated with poly-L-lysine (e.g., Sigma, Inc.), for example, by hand or using a microarrayer. The RNAi spots can be affixed to the slide by, for example, subjecting the resulting product to drying at room temperature, at elevated temperatures or in a vacuum-dessicator. The length of time necessary for sufficient drying to occur depends on several factors, such as the quantity of mixture placed on the surface and the temperature and humidity conditions used.

The concentration of RNAi present in the mixture will be determined empirically for each use, but will generally be in the range of from about 0.01 μg/μl to about 0.2 μg/μl and, in specific embodiments, is from about 0.02 μg/μl to about 0.10 μg/μl. Alternatively, the concentration of DNA present in the mixture can be from about 0.01 μg/μl to about 0.5 μg/μl, from about 0.01 μg/μl to about 0.4 μg/μl and from about 0.01 μg/μl to about 0.3 μg/μl. Similarly, the concentration of gelatin, or another carrier macromolecule, can be determined empirically for each use, but will generally be in the range of 0.01% to 0.5% and, in specific embodiments, is from about 0.05% to about 0.5%, from about 0.05% to about 0.2% or from about 0.1% to about 0.2%. The final concentration of RNAi in the mixture (e.g., RNAi in gelatin) will generally be from about 0.02 μg/μl to about 0.1 μg/μl and in a specific embodiment described herein, RNAi is diluted in 0.2% gelatin (gelatin in water) to produce a final concentration of RNAi equal to approximately 0.05 μg/μl.

While the above-described embodiment has been described in terms of “gelatin,” gelatin or an equivalent thereof may be employed. For example, in certain embodiments, the carrier is a hydrogel, such a polycarboxylic acid, cellulosic polymer, polyvinylpyrrolidone, maleic anhydride polymer, polyamide, polyvinyl alcohol, or polyethylene oxide.

In yet other embodiments, a RNAi-containing mixture (referred to herein as a lipid-RNAi mixture) which comprises RNAi; a carrier protein (e.g., gelatin); a sugar, such as sucrose; a buffer that facilitates RNAi condensation and an appropriate lipid-based transfection reagent is spotted onto a surface, such as a slide, thus producing a surface bearing the lipid-RNAi mixture in defined locations. The resulting product is allowed to dry sufficiently that the spotted lipid-RNAi mixture is affixed to the slide and the spots remain in the locations to which they have become affixed, under the conditions used for subsequent steps in the method. For example, a lipid-RNAi mixture is spotted onto a slide, such as a glass slide coated with poly-L-lysine (e.g., Sigma, Inc.), for example, by hand or using a microarrayer. The RNAi spots can be affixed to the slide as described above for the gelatin-RNAi method.

The concentration of RNAi present in the mixture will be determined empirically for each use, but will generally be in the range of 0.5 μg/μl to 1.0 μg/μl. A range of sucrose concentrations can be present in the mixture, such as from about 0.1M to about 0.4M. Similarly, a range of gelatin concentrations can be present in the mixture, such as from about 0.01% to about 0.05%. In this embodiment, the final concentration of DNA in the mixture will vary and can be determined empirically. In specific embodiments, final DNA concentrations range from about 0.1 μg/μl to about 2.0 μg/μl. If a vector is used in this embodiment, it can be any vector, such as a plasmid, or viral-based vector, into which DNA of interest (DNA to be expressed in reverse transfected cells) can be introduced and expressed (after reverse transfection), such as those described for use in the gelatin-DNA embodiment.

The total number of RNAi spots on the substrate will vary depending on the number of different RNAi agents to be explored or assayed, as well as the number of control spots, calibrating spots and the like, as may be desired. Generally, the pattern present on the surface of the support will comprise at least about 10 distinct spots, usually at least about 200 distinct spots, and more usually at least about 500 distinct spots, where the number of spots can be as high as 50,000 or higher, but will usually not exceed about 25,000 distinct spots, and more usually will not exceed about 15,000 distinct spots. Each distinct RNAi agent composition may be present in duplicate or more (usually, at least 5 replicas) to provide an internal correlation of results.

The amount of RNAi agent present in each spot will be sufficient to provide for adequate gene silencing in cells during the assay in which the array is employed. The spot will usually have an overall circular dimension and the diameter will range from about 10 to 5,000 μm, usually from about 20 to 1000 μm and more usually from about 50 to 500 μm. The RNAi agent will be present in the solution at a concentration of from about 0.0025 to about 10 μg/ml, and may be diluted in series to determine binding curves, etc.

In the subject arrays, the RNAi agent is not covalently bound to the surface of the substrate in many embodiments. As such, the RNAi is physically positioned on the surface, but not covalently bound to the surface.

The RNAi arrays of certain embodiments of the present invention are analogous to the DNA transfection arrays disclosed in U.S. application Publication No. 20020006664 (the disclosure of which is herein incorporated by reference); such that DNA arrays described in this application which are modified to have RNAi agents instead of DNA on the arrays fall within the scope of this invention.

Methods

The subject RNAi arrays, as described above, find use in transfection applications, such as modifications of the reverse transfection applications described in U.S. application Publication No. 20020006664 (the disclosure of which is herein incorporated by reference).

In practicing the subject methods, the subject RNAi arrays are contacted with a cellular population made up of a plurality of distinct cells (e.g., a suspension of cells), where the population is typically homogenous with respect to the nature of its constituent cells, such that all of the cells in the cell population contacted with the array are of the same type.

The type of cell that is contacted with the array may vary greatly, both in terms of species of origin and function. In many embodiments, the cells are from species that are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In certain embodiments, the cells will be human cells. Other types of cells include, but are not limited to: other animal cells, e.g., insects, invertebrates, and the like.

Cell types that can find use in the subject invention include stem and progenitor cells, e.g., embryonic stem cells, hematopoietic stem cells, mesenchymal stem cells, neural crest cells, etc., endothelial cells, muscle cells, myocardial, smooth and skeletal muscle cells, mesenchymal cells, epithelial cells; hematopoietic cells, such as lymphocytes, including T-cells, such as Th1 T cells, Th2 T cells, Th0 T cells, cytotoxic T cells; B cells, pre-B cells, etc.; monocytes; dendritic cells; neutrophils; and macrophages; natural killer cells; mast cells;, etc.; adipocytes, cells involved with particular organs, such as thymus, endocrine glands, pancreas, kidney, brain, such as neurons, glia, astrocytes, dendrocytes, etc. and genetically modified cells thereof. Hematopoietic cells may be associated with inflammatory processes, autoimmune diseases, etc., endothelial cells, smooth muscle cells, myocardial cells, etc. may be associated with cardiovascular diseases; almost any type of cell may be associated with neoplasias, such as sarcomas, carcinomas and lymphomas; liver diseases with hepatic cells; kidney diseases with kidney cells; etc.

The cells may also be transformed or neoplastic cells of different types, e.g. carcinomas of different cell origins, lymphomas of different cell types, etc. The American Type Culture Collection (Manassas, Va.) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCl (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

Cells may be non-adherent, e.g. blood cells including monocytes, T cells, B-cells; tumor cells, etc., or adherent cells, e.g. epithelial cells, endothelial cells, neural cells, etc. In order to employ adherent cells, the cells are typically dissociated from the substrate that they are adhered to, and from other cells, in a manner that maintains their viability. Methods of dissociating cells are known in the art, including protease digestion, etc. In certain embodiments, the dissociation methods use enzyme-free dissociation media.

Cell types of interest also include, but are not limited to, those described in U.S. application Publication No. 20020006664 (the disclosure of which is herein incorporated by reference).

The cells are contacted with the RNAi array, e.g., plated onto the array, under conditions sufficient for the cells to be transfected with the RNAi agents of the array. As such, the cells are placed or plated on the array surface, typically in the form of a monolayer, such that the cells over each given feature of the array take up the RNAi agent of the given feature and are thereby transfected by the RNAi agent of a given feature. In other words, the cells are contacted with the array under transfecting conditions.

In many embodiments the cells are contacted with the array as an aqueous suspension of the cells, where an agent that promotes uptake of the RNAi agent, such as a transfection reagent, e.g., (e.g., Effectine (Qiagen)) may be included.

The host cells are plated (placed) onto the surface bearing the transfection array in sufficient density and under appropriate conditions for introduction/entry of the nucleic acid into the cells. Preferably, the host cells (in an appropriate medium) are plated on the array at high density (e.g., on the order of 0.5-1×10⁵/cm²), in order to increase the likelihood that transfection will occur. For example, the density of cells can be from about 0.3×10⁵/cm² to about 3×10⁵/cm², and in specific embodiments, is from about 0.5×10⁵/cm² to about 2×10⁵/cm² and about 0.5×10⁵/cm² to about 1×10⁵/cm². The appropriate conditions for introduction/entry of DNA into cells will vary depending on the quantity of cells used.

Following contact or plating of the cells onto the array surface, the resultant cells are maintained on the surface under suitable conditions and for a sufficient period of time for the cells to be transfected by the various RNAi agents. Typically, the resultant cells plated on the array surface are maintained at a temperature ranging from about 20 to about 40, such as from about 25 to about 40° C., for a period of time for one or more, e.g., one to five, including one to three, e.g., two cell cycles to occur.

Representative plating protocols suitable for use in the subject methods are additionally described in U.S. application Publication No. 20020006664 (the disclosure of which is herein incorporated by reference).

After sufficient time has elapsed, slides are assessed for transfection (entry of RNAi into cells) and/or effect of the introduced RNAi agent on transfected cells, e.g., by using known methods. In many embodiments, cells positioned over each array feature or spot of RNAi are assayed or evaluated for any phenotypic variation from the wild-type phenotype.

Various cellular outputs may be assessed to determine the response of the cells to the input RNAi, including calcium flux, BrdU incorporation, expression of an endogenous or a transgene reporter, metabolic reporters, electrical activity (e.g. via voltage-sensitive dyes), release of cellular products, cell motility, size, shape, viability and binding, etc. Generally the analysis provides for site-specific determination, i.e., the cells that are localized at a spot are analyzed for phenotype in an individual or spot specific manner, which correlates with the spot to which the cells are localized.

The phenotype of the cell in response to a signaling probe or a microenvironment may be detected through changes in cell various aspects, usually through parameters that are quantifiable characteristics of cells. Characteristics may include cell morphology, growth, viability, expression of genes of interest, interaction with other cells, and include changes in quantifiable parameters, parameters that can be accurately measured.

A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. Parameters may provide a quantitative readout, in some instances a semi-quantitative or qualitative result. Readouts may include a single determined value, or may include mean, median value or the variance, etc. Variability is expected and a range of values for each of the set of test parameters will be obtained using standard statistical methods with a common statistical method used to provide single values.

Parameters of interest include detection of cytoplasmic, cell surface or secreted biomolecules, frequently biopolymers, e.g. polypeptides, polysaccharides, polynucleotides, lipids, etc. Cell surface and secreted molecules are a useful parameter type as these mediate cell communication and cell effector responses and can be readily assayed.

As such, a variety of methods can be used to detect the consequence of uptake of the RNAi agent. In a general sense, the assay provides the means for determining if the RNAi agent is able to confer a change in the phenotype of the cell relative to the same cell but which lacks the RNAi agent. Such changes can be detected on a gross cellular level, such as by changes in cell morphology (membrane ruffling, rate of mitosis, rate of cell death, mechanism of cell death, dye uptake, and the like). In other embodiments, the changes to the cell's phenotype, if any, are detected by more focused means, such as the detection of the level of a particular protein (such as a selectable or detectable marker), or level of mRNA or second messenger, to name but a few. Changes in the cell's phenotype can be determined by assaying reporter genes (beta-galactosidase, green fluorescent protein, beta-lactamase, luciferase, chloramphenicol acetyl transferase), assaying enzymes, using immunoassays, staining with dyes (e.g. DAPI, calcofluor), assaying electrical changes, characterizing changes in cell shape, examining changes in protein conformation, and counting cell number. Other changes of interest could be detected by methods such as chemical assays, light microscopy, scanning electron microscopy, transmission electron microscopy, atomic force microscopy, confocal microscopy, image reconstruction microscopy, scanners, autoradiography, light scattering, light absorbance, NMR, PET, patch clamping, calorimetry, mass spectrometry, surface plasmon resonance, time resolved fluorescence. Data could be collected at single or multiple time points and analyzed by the appropriate software.

Additional representative phenotypic evaluation protocols that may be employed in the subject methods are described in U.S. application Publication No. 20020006664 (the disclosure of which is herein incorporated by reference).

Detection of a phenotypic change in cells contacted with a given RNAi agent is then used to determine the activity of the RNAi agent with respect to expression of a gene to which it corresponds, as described above. Specifically, a change in a cell phenotype as compared to control observed in cells contacted with a given RNAi agent means that that RNAi agent has activity in modifying, and typically reducing, expression of the gene to which it corresponds.

Utility

The subject RNAi arrays and methods for using the same, as described above, find use in a number of different applications. One representative method of using the subject RNAi arrays is to determine the activity of two or more different RNAi agents with respect to one or more genes, and specifically the expression of one or more genes, of a given type of cell. In these embodiments, the impact of two or more different RNAi agents on the expression of one or more different genes in a given cell is determined at substantially the same, if not the same, time, e.g., as may be found in a high throughput format.

The above-described arrays and methods for using the same also find use in loss-of-function genetic assays, particularly in high-throughput formats of such assays. As such, the subject RNAi arrays can be used to assess the loss-of-function of a particular gene or genes. In such loss-of-function applications, an array of a plurality of different RNAi agents directed to one or more different genes is contacted with a cell suspension, as described above, under transfection conditions. Following transfection, the cells contacted with each different spot or feature are evaluated for phenotypic change, as described above. The location of cells exhibiting phenotypic variation of interest is then employed to determine the identity of the RNAi agent that transfected the cells of interest and caused the phenotypic change of interest. Identification of the RNAi agent is then used to determine the identity of the gene whose expression has been inhibited or reduced, resulting in the observed phenotype of interest. In this way, the function of the gene is extrapolated. In other words, the gene is annotated with respect to function.

Because arrays of RNAi agents are contacted with a plurality of cells and the resultant transfected cells are evaluated at the substantially the same, if not the same, time, the subject arrays and methods for using the same are particularly suited for use in high throughput loss of function genomic assays.

Representative utilities are also described in U.S. application Publication No. 20020006664 (the disclosure of which is herein incorporated by reference).

Kits

Also provided are reagents and kits thereof for practicing one or more of the above-described methods. The subject reagents and kits thereof may vary greatly. Typically, the kits at least include an RNAi array as described above, or components for producing the same, e.g., a substrate, gelatin, lipids, etc, vectors for use in siRNA production, enzymes, e.g., dicer, for use in siRNA production; etc.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### Experimental

### I. Materials and Methods

A. Cells and Reagents

Human embryonic kidney (HEK) 293 cells (American Tissue Culture Collection) and 293-derived Phoenix amphotropic packaging cell line (G. Nolan, Stanford) are obtained from the indicated sources. Single stranded dTdT RNA oligonucleotides (Dharmacon) were annealed to generate siRNA. Stable GFP-expressing Phoenix cells were produced by transient transfection of pMIGR (gift of W. Pear, U. Pennsylvania) into amphotropic Phoenix cells and followed by two rounds of fluorescence-activated cell sorting (FACS) selection of GFP+cells. The resultant cells were >95% GFP+ and remained so subsequently without additional selection. Constructs: eGFP-N3, dsRED, YFP-actin, and pSEAP2-control (Clontech), and pGL3 luciferase (Promega) were obtained from indicated sources. The Xhol-BamHl actin fragment from YFP-actin was released by restriction digestion and cloned into eGFP-N3 and pGL3-control to generate ActinS-GFP, ActinAS-GFP and Luciferase-actin constructs.

B. siRNA Experiments

Expression constructs and siRNAs were transfected using Lipofectamine 2000 (Invitrogen) as described in Elbashir et al., Nature (2001) 411:494-498. GFP expression was assayed by either FACS or fluorescence microscopy 48-72 hours after transfection. Transfection efficiency was normalized by dividing GFP or luciferase fluorecent units with the secreted placental alkaline phosphatase activity generated from cotransfected pSEAP2-control plasmid.

C. Microarray Procedures

Messenger RNA was purified using Fastrack (Invitrogen) following manufacturer's instructions. A reference mRNA standard prepared by pooling RNA from eleven cell lines were used in all experiments. Microarray techniques were as described Perou et al., Nature (2000) 406:747-752. For the siRNA arrays, the annealed RNA duplexes were precipitated in ethanol and resuspended in water for array printing. Complementary DNA and siRNA were dissolved in 0.2% gelatin and printed on amine-covered glass slides (Corning) using a robotic arrayer, and reverse trransfection of HEK293 cells was performed using Effectene (Qiagen) as described Ziauddin & Sabatini, Nature (2001) 411:107-110. Reverse transfected cells were visualized by digital phase contrast and fluorescence microscopy (Canon).

D. Statistical Methods

The gene expression data from 3 sets of siRNA experiments were derived from 27 microarrays and were analyzed separately in 3 data sets. In each data set, genes were considered well-measured if the reference channel had >1.5 fold of signal intensity over background and was present for >80% of data set. The three sets of genes were each analyzed by multi-class comparison in SAM Tusher et al., Proc. Nat'l Acad. Sci. USA (2001) 98:5116-5121, and the false discovery rate of the top 10 SAM-selected genes was calculated. The top 10 genes from each data set were collated, and the expression data of this set of 30 genes from each data set was retrieved and grouped by hierarchical clustering Eisen et al., Proc. Nat'l Acad. Sci. USA (1998) 95:14863-14868.

E. Silencing of a Model Gene by siRNAs

Silencing of transiently expressed and integrated GFP gene by siRNAs. Sequences of the siRNAs used were:

| | | | |
|---|---|---|---|
| 5′ | CUACAACAGCCACAACGUCdTdT | 3′ | (SEQ ID NO:01) |
| | dTdTGAUGUUGUCGGUGUUGCAG | | |
| 5′ | CAACAUCUCGACACCAGCAdTdT | 3′ | (SEQ ID NO:02) |
| | dTdTGUUGUAGAGCUGUGGUCGU | | |
| 5′ | CAGCCACAACGUCUAUAUCdTdT | 3′ | (SEQ ID NO:03) |
| | dTdTGUCGGUGUUGCAGAUAUAG | | |
| 5′ | ACAGACCACCGUGUCUAACdTdT | 3′ | (SEQ ID NO:04) |
| | dTdTUGUCUGGUGGCACAGAUUG | | |

For silencing of transiently transfected GFP, 0.3 μg of pGFP was transfected with 1 μg of pSEAP2-control and 12 picomoles of the indicated siRNA in HEK293 cells. For silencing of an integrated GFP gene, HEK293-derived Phoenix cells expressing GFP after retroviral transduction (Methods) were transfected with the 12 picomoles of the indicated siRNA and 1 μg of pSEAP2-control. GFP expression was determined by FACS 48 hours (transient GFP target) or 72 hours (integrated GFP target) after transfection. The mean fluorescence intensity was normalized for transfection efficiency by the alkaline phosphatase activity of pSEAP2-control (Methods). The experiments were done in triplicate, and the means (+standard deviation) of GFP fluorescence intensity relative to mock transfected cells (no siRNA) are shown. Fluorescence photomicroscopy and FACS plots of cells stably expressing GFP and transfected with the indicated siRNAs were also obtained.

F. Global Gene Expression Changes Associated with RNAi.

Global gene expression patterns in 3 siRNA experiments were analyzed; in each set the gene expression of cells which were mock transfected (no siRNA), transfected with GFP siRNA, or cognate control siRNA were determined in parallel in triplicate. Data sets: (E1) HEK293 cells with transiently expressed GFP target treated with E1, C1, or no siRNA; (E2) HEK293 cells with transiently expressed GFP target treated with E2, C2, or no siRNA; (stable) Phoenix cells stably expressing an integrated GFP gene treated with E1, C1, or no siRNA. Genes that had signal intensity >1.5 fold of the local spot element background in the reference channel and were present for >80% of the data set were considered well measured. A summary of the results is provided below:

| | Number of Well- | Number of Genes | |
|---|---|---|---|
| Data Set | Measured Genes | with FDR < 0.05 | FDR for top 10 genes |
| E1 | 17,891 | 0 | 0.19 |
| E2 | 24,048 | 0 | 0.30 |
| Stable | 19,655 | 0 | 0.22 |

The number of well-measured genes are shown on the second column; these genes were analyzed in the multi-class comparison using SAM. The number of genes which had an estimated false discovery rate (FDR) of <0.05 and the FDR of the top 10 performing genes for each data set are shown on the right two columns, Minimal gene expression changes associated with siRNA-mediated RNAi were observed. The 10 genes with the most consistent changes in expression in response to the experimental manipulation, in each of the 3 siRNA experiments, were collated into a non-redundant gene list. The expression changes of this group of genes in all experiments were displayed in matrix format. The expression ratios were mean-centered within each data set.

### II. Results

A. Global View of Gene Silencing by siRNA

To evaluate the specificity of siRNA, we used a target gene that has no normal role or known physiological effects in the cell, so that its presence or absence would not otherwise perturb the transcriptome. We chose the enhanced green fluoresecent protein (GFP) of Aequoria victoria as a model target because the protein level is easily monitored, it is an exogenous protein that has no normal function in human cells, and it is relatively nontoxic and known to be well tolerated in normal development. As previously reported by Elbashir et al., Nature (2001) 411: 3494498, transient transfection of HEK293 cells with GFP and the two siRNAs directed toward GFP sequences (termed E1 and E2) suppressed the level of GFP activity by over 80%, but cotransfection of GFP with scrambled siRNAs matched for nucleotide content (termed C1 and C2, respectively) did not affect GFP activity compared to mock transfected cells, which were not exposed to siRNA. C1 and C2 did not have significant homology to any human gene or expressed sequence tags (EST) in the NR and EST database when analyzed with Blast program in NCBI. The transfection efficiency was above 80% as judged by GFP fluorescence. To address the specificity of RNAi against an integrated and nuclear gene, we established a population of cells stably expressing a GFP gene that was introduced by retroviral transduction (Methods). Transfection of these stable GFP-expressing cells with the E1 siRNA silenced GFP expression by more than 70%, but GFP expression was unaffected by mock or C1 transfection.

The global gene expression patterns of cells after mock transfection, silencing of transiently expressed or stably expressed GFP by E1 or E2 siRNA, and control silencing by C1 or C2 siRNA were determined using human cDNA microarrays. The microarrays contained approximately 43,000 elements, corresponding to approximately 36,000 genes based on Unigene-data. Because even small differences in cell passage or media metabolism can lead to differences in global gene expression pattern, control and siRNA experiments were always performed in parallel in sets of three and in triplicate as described above. To search for gene expression responses associated with RNA interference, we performed a statistical test (SAM) to identify genes whose expression varied accordingly in response to the experimental manipulations we tested, Tusher et al., supra. SAM is a permutation-based technique that permits the estimation of a false discovery rate (FDR) for set of genes identified Tusher et al., supra. The FDR is analogous to p-value in standard statistical tests, but the FDR can accommodate the effects of non-normal distribution in the data and multiple testing Tusher et al., supra. For each of the three sets of gene expression data, none of approximately 20,000 well-measured mRNAs was consistently affected by the siRNA treatments, with a FDR <0.05 . The 10 genes that showed the most consistent changes in expression with the experimental manipulations had estimated FDRs that ranged from 0.19 to 0.30 in the three experiments. The top 10 genes identified by SAM in all three data sets were noted. We note that the genes that showed the largest apparent responses in the three sets of experiments did not overlap, and the magnitude of the changes in expression of any of these genes was small (mostly less than 2 fold).

Moreover, these small variations in gene expression did not consistently distinguish the siRNA-silenced samples from the mock treated samples. Among all of the genes that showed variation in expression in the experiments identifying either transiently or stably GFP, none showed a consistent response pattern. Thus, we believe that the small observed variations are likely to be due to experimental noise, rather than resulting from the siRNA treatment.

Collectively, we interpret these results to indicate that no consistent “off-target” gene expression perturbation is associated with the process of siRNA-mediated gene silencing. To the detectable limits of our cDNA array method, siRNA-mediated gene silencing in the tested cells appears to be highly sequence-specific.

B. Evaluation of Transitive RNAi in Human Cells

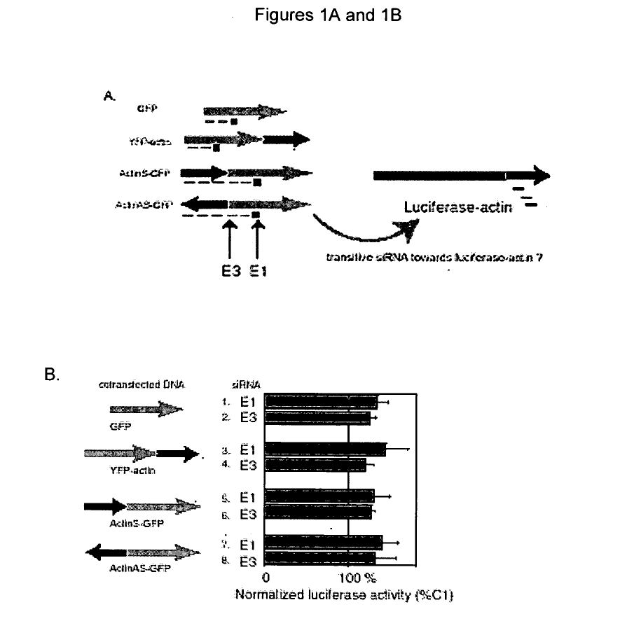

Although siRNAs appear to be highly sequence-specific, the extension of RNAi-mediated silencing to sequences 5′ to the mRNA sequence complementary to the siRNA could generate secondary siRNAs that could potentially target other mRNAs with sequence similarity. Such a phenomenon, termed “transitive RNAi” has been shown to occur in C. elegans (Sijen et al., Cell (2001) 107:465-476. To test for the occurrence of transitive RNAi in human cells, we cotransfected into HEK 293 cells two sets of reporter genes (GFP/YFP and luciferase) with sequence overlap engineered by fusing a sequence for the actin gene to both sets of constructs (FIG. 1A). We used siRNA E1 to target the first reporter genes (GFP or YFP, which contain the same cognate sequence) and verified the RNA silencing by monitoring the fluorescence of transfected HEK293 cells. If transitive RNAi were active in 293 cells, silencing of GFP/YFP-actin fusion mRNA should generate secondary siRNAs targeting the actin sequences and thereby initiate the silencing of the second reporter gene, luciferase-actin, resulting in diminished luciferase activity. We tested the transitive effects of silencing GFP expressed alone or in the form of fusion transcripts with actin fused at either the 3′ end of yellow fluorescent protein (YFP-actin), or at the 5′ end of GFP in both orientations (ActinS-GFP, ActinAS-GFP). Fluorescent microscopy confirmed that siRNA-mediated RNA silencing of the primary target gene was achieved for all four pairs of different fluorescent proteins. In all four experiments, the luciferase activity in the cells silenced by GFP siRNA (E1) was not lower than that in cells treated with control siRNA (C1) (FIG. 1B). These results show that transitive RNAi, at least on the scale demonstratable in Drosophila extract and C. elegans, does not occur during siRNA-mediated silencing in 293 cells. This result may be related to the relatively inefficient silencing mediated by siRNA in mammalian cells compared to that seen in Drosophila or C. elegans.

C. siRNA-mediated RNAi on Microarrays

The rapidly expanding catalogue of eukaryotic genes, from a diverse and expanding array of sequencing projects, presents scientists with the challenge of understanding the biological roles of each newly identified gene. Recent advances in RNAi technology in lower organisms have already yielded powerful insights into the functions of many genes and their protein products. RNAi has been successfully applied to systemic analysis of the C. elegans genome, but the effort still depends on the analysis of the phenotypes of individual worms resulting from disruption of one gene at one time. The recent development of high-throughput cDNA transfection on microarrays (Ziauddin & Sabatini et al., supra) provides a model for the use of siRNAs on high-density microarrays to perform RNAi in mammalian cells in a highly parallel fashion.

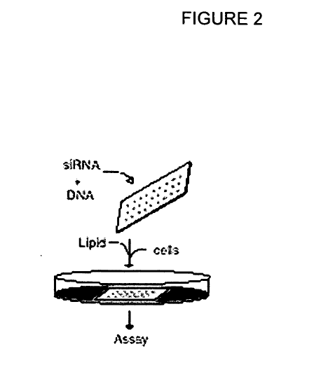

We tested the feasibility of siRNAs-mediated RNAi on microarrays (FIG. 2). DNAs encoding GFP, dsRED, and siRNAs were spotted in the desired combinations on amine glass slides using a robotic arrayer. We hypothesized that in the presence of lipids, siRNA would complex with the DNA printed on the slide and form liposomes containing both reagents. Expression of dsRED served as an internal control for reverse transfection and localization of the printed spots. After air drying, the printed arrays were exposed to Effectene briefly and placed in a tissue culture dish. HEK293 cells were then plated on the arrays and cultured in Petri dish. The cells were examined with fluorescence microscopy 72 hour later. As shown in FIG. 4B, HEK293 cells expressed dsRED in all the cell clusters above the printed spots after reverse transfection. In contrast, GFP expression was readily apparent in the control spots and selectively decreased in the presence of the siRNA E1, complimentary to GFP mRNA, but not in the presence of the control siRNA C1 (data not shown). The merged image allowed quick detection of specific RNAi effect by the red shift of the affected cell clusters.

These results demonstrate that siRNA-mediated gene silencing can be adapted to microarray format. By arraying different siRNAs on microarrays, one can generate a large panels of cells silenced for different genes for highly parallel tests of gene function.

### III. Discussion

Using DNA microarrays to profile global gene expression, we have demonstrated that siRNA-mediated gene silencing has exquisite sequence specificity for the target mRNA and does not induce detectable secondary changes in the global gene expression pattern. We tested for transitive RNAi using paired, highly-expressed transcripts with overlapping sequence identity, conditions which easily afforded detection of transitive RNAi in C. elegans (Sijen et al., supra). The lack of robust transitive RNAi in human cells is consistent with published reports of selective targeting of splicing isoforms using siRNA, the lack of an obvious RNA-dependent RNA polymerase in the human genome, and the dispensability of priming activity of siRNAs for RNAi in mammalian cells. These results provide further confirmation for using siRNA-mediated RNAi as a research and therapeutic tool. The high specificity observed in these experiments, increases the confidence with which phenotypes observed with siRNA-mediated silencing can be ascribed to the targeted genes. The results confirm the position that siRNA-based therapeutic agents have useful molecular specificity. Because the process of siRNA-mediated silencing does not appear, in general, to produce nonspecific gene expression changes, global changes of gene expression patterns provide an assay with which to study and annotate the function of unknown genes, especially based on comparisons to gene expression patterns of mutants in known pathways (Hughes et al., Cell (2002) 102: 109-126).

Application of siRNA technology on a genome-wide scale could be significantly accelerated by a platform for delivering siRNAs and screening the resulting phenotypes in a high throughput fashion. We have examined the feasibility of arraying siRNAs on glass microarrays and performing RNAi experiments by reverse transfection. This method provides a practical means to conduct highly parallel RNAi experiments in mammalian cells in a spatially-addressable fashion. Approximately 10,000 array elements can be accommodated on a standard glass microscope slide in the format that we tested. As we have demonstrated using two reporter genes to monitor transfection and gene silencing separately, arrays of cells silenced for different genes may be screened for altered morphology, activation of signal transduction pathways using specific reporter genes, or expression of endogenous markers using immunofluorescence. The microarray format also lends itself to comprehensively testing the effect of silencing various combinations of genes within a family and thereby confronting the issues of redundancy and compensation that frequently arise in mammalian genetics.

It is evident from the above results and discussion that the subject invention provides an important new way of performing RNAi mediated loss of function assays. Specifically, the subject invention provides high throughput formats for such assays. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of determining the activity of two or more RNAi agents, said method comprising:
(a) contacting a population of cells with an array comprising said two or more RNAi agents; and
(b) evaluating said contacted cells to determine the activity of said two or more RNAi agents.

2. The method according to claim 1, wherein said RNAi agents are double-stranded RNA molecules.

3. The method according to claim 1, wherein said RNAi agents are short hairpin RNA molecules.

4. The method according to claim 1, wherein said RNAi agents comprise a duplex structure that ranges in length from about 20 to 30 bp.

5. The method according to claim 1, wherein said array comprises a planar support having said two or more RNAi agents positioned in known and discrete locations on a surface thereof.

6. The method according to claim 1, wherein said evaluating comprises assessing at least one phenotypic characteristic of said cells.

7. The method according to claim 1, wherein said cells are mammalian cells.

8. The method according to claim 7, wherein said mammalian cells are human cells.

9. A method of assaying the activity of two or more genes, said method comprising:
(a) contacting a population of cells with an array comprising a distinct RNAi agent for each of said genes; and
(b) evaluating said contacted cells to assay the activity of said two or more genes.

10. The method according to claim 9, wherein said RNAi agents are double-stranded RNA molecules.

11. The method according to claim 9, wherein said RNAi agents are short hairpin RNA molecules.

12. The method according to claim 9, wherein said RNAi agents comprise a duplex structure that ranges in length from about 20 to 30 bp.

13. The method according to claim 9, wherein said array comprises a planar support having said two or more RNAi agents positioned in known and discrete locations on a surface thereof.

14. The method according to claim 9, wherein said evaluating comprises assessing at least one phenotypic characteristic of said cells.

15. The method according to claim 9, wherein said cells are mammalian cells.

16. The method according to claim 15, wherein said mammalian cells are human cells.

17. An array comprising two or more RNAi agents.

18. The array according to claim 17, wherein said array comprises a planar support with said two or more RNAi agents present on a surface thereof in discrete and known locations.

19. The array according to claim 17, wherein said array comprises at least about 50 distinct RNAi agents.

20. The array according to claim 17, wherein said RNAi agents are double-stranded RNA molecules.

21. The array according to claim 17, wherein said RNAi agents are short hairpin RNA molecules.

22. The array according to claim 17, wherein said RNAi agents comprise a duplex structure that ranges in length from about 20 to 30 bp.

23. The array according to claim 17, further comprising a layer of cells on said surface.

24. A kit comprising:
an RNAi array of two or more RNAi agents; and
instructions for practicing the method of claim 1.

25. The kit according to claim 24, wherein said kit further comprises a transfectant reagent.
